(19) United States
(12) Patent Application Publication
(10) Pub. No.: US 2009/0311202 A1
(43) Pub. Date: Dec. 17, 2009

(54) COSMETIC PREPARATION FOR EXTENSION TANNING

(76) Inventors: Karin Golz-Berner, Monaco (MC); Leonhard Zastrow, Monaco (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/722,001

(22) PCT Filed: Dec. 6, 2005
(86) PCT No.: PCT/EP2005/013197

§ 371 Date: Mar. 2, 2009

(30) Foreign Application Priority Data
Dec. 17, 2004 (DE) 10 2004 062 170.5

(51) Int. Cl. A61K 8/49; A61Q 17/04

(52) U.S. Cl. 424/59

(57) ABSTRACT

The invention relates to a cosmetic preparation for tan extension. The cosmetic includes as active ingredient a complex of 0.1 to 3.0 wt.-% caffeine, 0.2 to 2 wt.-% Phototan®, 0.01 to 1.0 wt.-% copper gluconate and further cosmetic auxiliaries, excipients, further active substances and mixtures thereof to make 100 wt.-%. It is possible to achieve a sun tan extension of up to 7 to 9 days, and in some cases up to 11 days.

The invention relates to a cosmetic preparation used to prolong the skin tan resulting from natural sun radiation.

Phototan® LS 2261 E is a well-known agent used to stimulate the enzymatic activity of dopa oxidase and accelerates the formation of epidermal melanin pigments. For this reason, it has been used as an accelerator of the sun tanning process and as sun tan enhancer.

Phototan® consists of a mixture of sorbitol, arginine-HCl, ornithine-HCl, tyrosine and silica gel. According to the manufacturer's instructions, the above mixture is to be used at a concentration of 3 to 5 wt.-% and is also capable of enhancing an existing sun tan, the maximum however being about 3-4 days.

The invention is based on the object of maintaining a substantial percentage of an existing sun tan for more than 3-4 days, thus achieving long-lasting effectiveness.

According to the invention, said object is accomplished by means of a sun tan extender according to claim 1, which includes as active ingredient a complex of

0.1 to 3.0 wt.-% caffeine,

### 0.2 to 2 wt.-% Phototan®,

0.01 to 1.0 wt.-% copper gluconate
and further cosmetic auxiliaries/excipients, further active substances and mixtures thereof to make 100 wt.-%, each relative to the overall weight of the preparation.

Advantageous embodiments of the invention are set forth in the subclaims.

It was found that a combination of the above-mentioned substances in a suitable formulation in the form of an emulsion or a gel results in sun tan extension and, as a consequence, tan enhancement of up to 7-9 days.

Such an enhancing effect was not to be expected by the substances employed in the above combination, such as caffeine and copper gluconate.

It is well-known that caffeine has some blood flow-stimulating effect and can be used in beauty culture for the treatment of acne, cellulitis and loss of hair.

Copper gluconate has been used as anti-inflammatory agent in cell regeneration, as immunomodulator and as activator of lysyl oxidase for collagen synthesis, and as cofactor of tyrosinase for melanin synthesis. Copper gluconate has some tanning-active properties which, however, when taken together alone, are too weak to induce significant tan enhancement.

The amino acid glycine, which has been used in cosmetics, e.g. in skin and hair conditioners, can be employed as further active substance in the preparation according to the invention. Another embodiment of the invention additionally includes a fruit extract of Citrus aurantium. Both additives can prolong the sun tan enhancement to up to 11 days, thus representing a preferred embodiment of the invention.

Particularly preferred cosmetic moisturizing agents for the preparation of the invention are propylene glycol, glycerol and e.g. Lubragel® and mixtures thereof.

Plant extracts, vitamins, flavones, flavonoids can be used as active substances with free radical-scavenging properties.

Particularly preferred is a mixture of plant extracts including as free radical-scavenging component 0.4 to 1.5 wt.-% of an alcohol-based plant extract mixture consisting of extracts of green coffee beans, angelica roots, Camellia sinensis leaves and Pongamia pinnata (WO 2004/105706).

Another additive for the cosmetic preparation of the invention is an active substance preparation according to WO99/66881 with a high free-radical protection factor and with a content of a product obtained by extraction of the bark of Que-bracho blanco and subsequent enzymatic hydrolysis, which product includes at least 90 wt.-% of proanthocyanidin oligomers and at most 10 wt.-% of gallic acid, in microcapsules, and a silkworm extract obtained by extraction, which includes the peptide cecropin, amino acids and a vitamin mixture, and a non-ionic, cationic or anionic hydrogel or a mixture of hydrogels, and one or more phospholipids and water.

The content of Phototan® can preferably range from 0.4 to 0.8 wt.-%.

The content of the free radical-scavenging component (plant extract of green coffee beans, angelica roots, Camellia sinensis leaves and Pongamia pinnata) can preferably range from 0.4 to 1.5 wt.-%.

Particularly preferred is a caffeine content of from 0.1 to 2.0, more specifically from 0.1 to 0.5 wt.-%.

The preparation according to the invention may also include cosmetic auxiliaries and excipients, as usually employed in such preparations, e.g. water, preservatives, dyes, pigments with a coloring effect, thickeners, fragrances, alcohols, polyols, esters, electrolytes, gelling agents, polar and non-polar oils, polymers, copolymers, emulsifiers, waxes and stabilizers.

The cosmetic active substances include e.g. inorganic and organic sunscreen agents, free-radical scavengers, moisturizing agents, vitamins, enzymes, vegetable active substances, polymers, antioxidants, antiphlogistic natural active substances and softening agents.

For example, a preferred additional active substance is kaolin modified with spherical SiO₂ or TiO₂ according to WO 96/17588, Example 1, or one or more antioxidants, flavones, flavonoids or mixtures of active substances of this group.

The antioxidants and free radical-scavengers include vitamins such as vitamin C and derivatives thereof, e.g. ascorbyl acetate, phosphate and palmitate; vitamin A and derivatives thereof; folic acid and its derivatives, vitamin E and its derivatives such as tocopheryl acetate; flavones or flavonoids; amino acids such as histidine, glycine, tyrosine, tryptophan and derivatives thereof; carotenoids and carotenes such as α-carotene, β-carotene; uric acid and derivatives thereof; α-hydroxy acids such as citric acid, lactic acid, malic acid; stilbenes and derivatives thereof.

It is also advantageous to add appropriate water- and/or oil-soluble UVA or UVB filters or both to the compositions of the invention. The advantageous oil-soluble UVB filters include 4-aminobenzoic acid derivatives such as 2-ethylhexyl 4-dimethylaminobenzoate; esters of cinnamic acid such as 2-ethylhexyl 4-methoxycinnamate; benzophenone derivatives such as 2-hydroxy-4-methoxybenzophenone; 3-benzylidenecamphor derivatives such as 3-benzylidenecamphor.

Water-soluble UVB filters include, for instance, sulfonic acid derivatives of benzophenone or of 3-benzylidenecamphor or salts such as the Na or K salt of 3-phenylbenzimidazole-5-sulfonic acid.

The UVA filters include dibenzoylmethane derivatives such as 1-phenyl-4-(4′-isopropylphenyl)propane-1,3-dione, butylmethoxybenzoylmethane and menthyl anthranilate.

Particularly preferred are benzophenone-3, butylmethoxydibenzoylmethane, octyl methoxycinnamate, octyl salicylate, 4-methylbenzylidenecamphor, homosalates, octocrylene, ethylhexyl methoxycinnamate, isoamyl p-methoxycinnamate, octyl dimethyl PABA, ethylhexyltriazone, diethylhexylbutamidotriazone, ethylhexyl salicylate, methylene-bis(benzotriazolyl)tetramethylbutylphenol, disodium phenyl dibenzimidazole tetrasulfonate, bis-ethylhexyloxyphenol methoxyphenyltriazine.

Also usable as sun protection filters are inorganic pigments based on metal oxides, such as TiO₂, SiO₂, ZnO, Fe₂O₃, ZrO₂, MnO, Al₂O₃, which can also be used in a mixture.

Cosmetic compositions having the active substance preparation can be in the form of O/W or W/O emulsions. Suitable emulsifiers for O/W emulsions are e.g. addition products of 2-30 moles of ethylene oxide with linear C₈-C₂₂ fatty alcohols, with C₁₂-C₂₂ fatty acids and with C₈-C₁₅ alkylphenols; C₁₂-C₂₂ fatty acid mono- and diesters of addition products of 1-30 moles of ethylene oxide with glycerol.

Suitable emulsifiers for W/O emulsions are, for example, addition products of 2-15 moles of ethylene oxide with castor oil; esters of C₁₂-C2₂ fatty acids and glycerol, polyglycerol, pentaerythritol, sugar alcohols (e.g. sorbitol), polyglucosides (e.g. cellulose); polyalkylene glycols; wool wax alcohols; copolymers of polysiloxane-polyalkyl polyethers.

The oils used in the invention can be conventional cosmetic oils, such as mineral oil; hydrogenated polyisobutene; squalane produced synthetically or from natural products; cosmetic esters or ethers which can be branched or unbranched, saturated or unsaturated; vegetable oils; or mixtures of two or more thereof.

Especially suitable oils are, for example, silicone oils, mineral oils, hydrogenated polyisobutene, polyisoprene, squalanes, tridecyl trimellitate, trimethylpropane triisostearate, isodecyl citrate, neopentylglycol diheptanoate, PPG-15 stearyl ethers as well as vegetable oils, such as calendula oil, jojoba oil, avocado oil, macadamia nut oil, castor oil, cocoa butter, coconut oil, maize oil, cottonseed oil, olive oil, palm nut oil, rapeseed oil, safflower oil, sesame oil, soybean oil, sun-flower oil, wheat germ oil, grape seed oil, candlenut oil, thistle oil and mixtures thereof.

Depending on which oils are selected, the cosmetic properties of a solid composition are affected, such as degree of transparency, softness, hardness and spreading effect.

As softening agents, a variety of compounds can normally be employed, such as stearyl alcohol, glyceryl monoricinoleate, glyceryl monostearate, propane-1,2-diol, butane-1,3-diol, cetyl alcohol, isopropyl isostearate, stearic acid, isobutyl palmitate, isopropyl myristate, isopropyl palmitate, oleyl alcohol, isopropyl laurate, decyl oleate, octadecan-2-ol, isocetyl alcohol, cetyl palmitate, silicone oils such as dimethylpolysiloxane, polyethylene glycol, lanolin, cocoa butter, vegetable oils such as maize oil, cottonseed oil, olive oil, mineral oils, butyl myristate, palmitic acid etc.

Suitable moisturizing agents are, for example, glycerol, butylene glycol, polypropylene glycol and mixtures thereof.

As colored pigments, pigment mixtures or powders having a pigment-like effect, also including those having a nacreous effect, the following can be employed, for example: iron oxides, natural aluminum silicates such as ocher, titanium (di)oxide, mica, kaolin, manganese-containing clays such as umbra and red bolus, calcium carbonate, talc, mica titanium oxide, mica titanium oxide-iron oxide, mica titanium oxide-organic dye, and mixtures thereof.

The cosmetic compositions according to the invention can be used e.g. in the form of sun creams, sun gels, after-sun products, pre-sun products, day creams, night creams, body lotions, cleansing milk, body powder, eye beauty care, and in products of decorative beauty culture such as deo sticks, perfume sticks, lip sticks, gels, eye shadows, compact products such as compact powders or compact wax, rouge, grounding, makeup, etc. The production of such products is effected in a way well-known to a person skilled in this field.

The invention is also directed to the use of a cosmetic preparation containing 0.1 to 3.0 wt.-% caffeine, 0.2 to 2 wt.-% Phototan®, 0.01 to 1.0 wt.-% copper gluconate and further cosmetic auxiliaries/excipients, further active substances and mixtures thereof to make 100 wt.-%, each relative to the overall weight of the preparation, to be applied to skin tanned by natural sun radiation, which application is effected at least twice a day with a minimum interval of 10 hours.

In a preferred fashion, said use is effected together with an additional active substance, which active substance is selected among glycine or an extract of Citrus aurantium fruits.

The invention will be explained in greater detail below with reference to examples. All the details are given in weight percent unless otherwise stated.

Example 1
Tan enhancer cream
Phase A

| H₂O | q.s. ad 100 |
|---|---|
| Carbomer | 2.0 |
| Glycerol | 4.0 |

Phase B

| | |
|---|---|
| Steareth 2 | 4.0 |
| Steareth 21 | 3.0 |
| Cetearyl alcohol | 1.5 |
| Mineral oil | 1.5 |

Phase C

| | |
|---|---|
| Triethanolamine | 2.0 |

Phase D

| | |
|---|---|
| Phototan ® | 0.8 |
| Copper gluconate | 0.01 |
| Caffeine | 0.5 |

Phase E

| | |
|---|---|
| RPF complex* | 1.0 |

Phase F

| | |
|---|---|
| Perfume oil | 0.5 |
| Preservative | 0.5 |

*According to WO 99/66881, Example 2.

Phases A and B are produced separately at about 70° C. and are subsequently combined. After cooling the mixture to 50° C., phase C is added with stirring. After further cooling to about 35° C., phases D and E are added. Finally, phase F is added at about 30° C., and the mixture is homogenized for about 20 minutes.

Example 2
Lotion
Phase A

| H₂O | q.s. ad 100 |
|---|---|
| Glycerol | 4.0 |

Phase B

| | |
|---|---|
| PEG-20 methylglycose sesquistearate | 2.0 |
| Tocopherol | 3.2 |
| Petrolatum | 2.0 |
| Squalane | 6.0 |
| Silicone | 2.0 |

Phase C

| | |
|---|---|
| Phototan ® | 0.5 |
| Copper gluconate | 0.01 |
| Caffeine | 0.1 |
| Glycine | 0.01 |

Phase D

| | |
|---|---|
| RPF complex* | 1.0 |

Phase E

| | |
|---|---|
| Perfume | 0.5 |
| Preservative | 0.5 |

*According to WO 99/66881, Example 1.

The phases A and B, produced separately, are stirred together at about 65° C. Subsequently, the mixture is added with phase C at 35° C. and 800 rpm at maximum and with phases D and E at 30° C., and this is finally homogenized for several minutes.

Example 3
Night cream
Phase A

| H₂O | q.s. ad 100 |
|---|---|
| Carbomer | 0.1 |

Phase B

| | |
|---|---|
| Stearic acid | 2.0 |
| Cetearyl alcohol | 2.0 |
| Glyceryl stearate | 2.0 |

Phase C

| | |
|---|---|
| Triethanolamine | 0.1 |

Phase D

| | |
|---|---|
| Phototan ® | 0.4 |
| Copper gluconate | 0.01 |
| Caffeine | 2.0 |
| Glycine | 0.01 |
| Biotanning ®¹ | 1.0 |

Phase E

| | |
|---|---|
| RPF complex* | 0.5 |

Phase F

| | |
|---|---|
| Perfume oil | 0.5 |
| Preservative | 0.5 |

*According to WO 99/66881, Example 1.
¹INCI: Hydrolyzed Citrus aurantium dulcis fruit extract.

The procedure corresponds to that of Example 1.

| | |
|---|---|
| Example 4 | Comparative test |

Using a preparation according to the invention, tan extension was measured on a group of 10 female test subjects 18 to 43 years of age who had combination skin. The test subjects had about the same clearly visible skin tan as a result of natural sun tanning. The initial values and subsequent values were measured on the forearms using an MX18 Mexameter® (Courage+Khazaka electronic GmbH, Germany). The forearms of the test subjects were not exposed to any UV radiation during the test period of 14 days.

Each measuring point was used to determine 8 measured values for the melanin value. The test subjects applied the cosmetic preparations as follows:

test subjects 1 to 5: lotion of Example 2 in the morning and in the evening;
test subjects 6 to 10: cream of Example 1 in the morning and in the evening.

The following Table 1 represents the percent drop of the melanin values during the course of 14 days.

TABLE 1
Profile of melanin values (MX18 Mexameter ®) in %

Days

| S | 0 | 4 | 7 | 9 | 11 | 14 |
|---|---|---|---|---|---|---|
| 1 | 100 | 95 | 88 | 84 | 81 | 62 |
| 2 | 100 | 92 | 90 | 86 | 79 | 60 |
| 3 | 100 | 90 | 86 | 80 | 74 | 64 |
| 4 | 100 | 89 | 83 | 77 | 72 | 58 |
| 5 | 100 | 93 | 87 | 81 | 76 | 62 |
| 6 | 100 | 96 | 89 | 83 | 72 | 59 |
| 7 | 100 | 90 | 85 | 78 | 68 | 58 |
| 8 | 100 | 93 | 89 | 85 | 72 | 60 |
| 9 | 100 | 91 | 88 | 84 | 74 | 59 |
| 10 | 100 | 94 | 87 | 80 | 71 | 61 |
| C 1 | 100 | 88 | 79 | 70 | 62 | 52 |
| C 2 | 100 | 85 | 70 | 63 | 58 | 48 |
| C 3 | 100 | 84 | 68 | 60 | 49 | 43 |

S = subject No.;
C = control No.

As is clearly evident from the Table, the skin tan after 7 days still was clearly above 80% of the initial value, with an average of about 87%, while the control (application of a lotion or cream with no phase C of Example 2 or phase C of Example 1) was around 72%. Also, further extension by additional components in accordance with Example 2 shows mean values of about 76% after 11 days, while the control was around 56%. Apparently, this represents a significant tan extension.

1. A cosmetic preparation for tan extension, characterized in that it includes as active ingredient a complex of
0.1 to 3.0 wt.-% caffeine,
0.2 to 2 wt.-% Phototan®,
0.01 to 1.0 wt.-% copper gluconate
and further cosmetic auxiliaries, excipients, further active substances and mixtures thereof to make 100 wt.-%, each relative to the overall weight of the preparation.

2. The cosmetic preparation according to claim 1, characterized in that it includes 0.01 to 1.0 wt.-% of the amino acid glycine as further active substance.

3. The cosmetic preparation according to claim 1, characterized in that it includes as further active substance 0.001 to 0.002 wt.-% of an extract of Citrus aurantium fruits, preferably 0.001 to 0.0015 wt.-%.

4. The cosmetic preparation according to claim 1, characterized in that it includes free radical-scavenging components as further active substances.

5. The cosmetic preparation according to claim 1, characterized in that it includes as free radical-scavenging component 0.4 to 1.5 wt.-% of an alcohol-based plant extract mixture consisting of extracts of green coffee beans, angelica roots, Camellia sinensis leaves and Pongamia pinnata.

6. The cosmetic preparation according to claim 1, characterized in that it includes preferably 0.1 to 2.0, particularly 0.1 to 0.5 wt.-% caffeine.

7. Use of a cosmetic preparation containing 0.1 to 3.0 wt.-% caffeine, 0.2 to 2 wt.-% Phototan®, 0.01 to 1.0 wt.-% copper gluconate and further cosmetic auxiliaries, excipients, further active substances and mixtures thereof to make 100 wt.-%, each relative to the overall weight of the preparation, to be applied to skin tanned by natural sun radiation, which application is effected at least twice a day with a minimum interval of 10 hours.

8. The use according to claim 7, characterized in that said use is effected together with an additional active substance, which active substance is selected among glycine or an extract of Citrus aurantium fruits.

9. The use according to claim 8, characterized in that said use is effected together with an additional active substance, which active substance is 0.01 to 1.0 wt.-% of the amino acid glycine.

10. The use according to claim 8, characterized in that said use is effected together with an additional active substance, which active substance is 0.001 to 0.002 wt.-% of an extract of Citrus aurantium fruits, preferably 0.001 to 0.0015 wt.-%.

11. A method of extending tan comprising applying to skin tanned by natural sun radiation, which application is effected at least twice a day with
a minimum interval of 10 hours,
a cosmetic preparation of claim 1.
